(19) United States
(12) Patent Application Publication
(10) Pub. No.: US 2012/0196803 A1
(43) Pub. Date: Aug. 2, 2012

(54) FUSION PROTEINS FOR DELIVERY OF GDNF AND BDNF TO THE CENTRAL NERVOUS SYSTEM

(75) Inventors: Michel Demeule, Beaconsfield (CA); Dominique Boivin, Ste-Marthe-sur-le-lac (CA); Jean-Paul Castaigne, Mont-Royal (CA)

(73) Assignee: ANGIOCHEM INC., Montreal (CA)

(21) Appl. No.: 13/377,480

(22) PCT Filed: Jun. 11, 2010
(86) PCT No.: PCT/CA2010/000889

§ 371 Date: Apr. 9, 2012

Related U.S. Application Data
(60) Provisional application No. 61/186,246, filed on Jun. 11, 2009.

(51) Int. Cl. A61K 38/18; C12N 15/62; C12N 15/63; A61P 25/28; A61P 25/00; A61P 25/24; A61P 25/18; C07K 19/00; C12P 21/02

(52) U.S. Cl. 514/7.6; 530/350; 536/23.4; 435/320.1; 435/69.7

(57) ABSTRACT

The present invention relates to a compound that includes a peptide vector, such as angiopep-2 which acts as a carrier across the blood-brain barrier, linked to glial-derived neurotrophic factor (GDNF), brain-derived neurotrophic factor (BDNF), or a related molecule, such as an analog or a fragment thereof. The compounds of the invention may be used to treat any disease where increased neuronal survival or growth is desired, e.g., neurodegenerative diseases, such as Parkinson's disease or amyotrophic lateral sclerosis. Other diseases can be treated using the compounds include schizophrenia and depression.

### BACKGROUND OF THE INVENTION

The invention relates to a conjugate including a peptide vector and either glial-derived neurotrophic factor (GDNF) or brain-derived neurotrophic factor (BDNF) and uses thereof.

Diseases associated with loss of or damage to neurons are serious conditions and affect millions world-wide. While therapies such as GDNF hold promise in treating neurodegenerative disorders such as Parkinson's disease, prior to the present invention, delivery of such therapeutics to the brain was complicated by the inability of the active agent to cross the blood-brain barrier. Indeed, previous clinical trials involving GDNF therapy for Parkinson's disease required the use of direct injection of the agent into the brain, and BNDF trials for treatment of amyotrophic lateral sclerosis involved intrathecal injection of the agent. These methods can be cumbersome and difficult.

Because there is a need for therapeutic treatment for a wide variety of diseases in which increased neuronal survival or growth is beneficial, GDNF- and BDNF-based therapeutics possessing the ability to cross the blood-brain barrier are desirable.

### SUMMARY OF THE INVENTION

We have now developed compounds that include a peptide vector conjugated to GDNF, BDNF, or a related molecule. These compounds are exemplified by a fusion protein including a GDNF or BNDF sequence and the Angiopep-2 sequence. In certain embodiments, these compounds can cross the blood-brain barrier, and thus are useful as therapeutics in treating subjects having a neurodegenerative disease or a neuronal injury.

Accordingly, in a first aspect, the invention features a compound including the formula:

A-X-B

where A is peptide vector; B is a polypeptide substantially identical to (i)

GDNF, a fragment thereof having at least one GDNF activity, or a GDNF analog (e.g., any described herein); or (ii) BDNF, a fragment thereof having at least one BDNF activity, or a BDNF analog (e.g., any described herein); and X is a linker (e.g., any described herein) that joins A to B. The compound may be capable (e.g., efficiently) of crossing the blood-brain barrier. The compound may include a mature form of GDNF (e.g., amino acids 118-211 of isoform 1) or a mature form of BDNF (e.g., amino acids 129-247 of the isoform A). The GDNF fragment may include or may be amino acids 78-211 of isoform 1). The compound may further include a tag, such as a His tag or a cleavage site, such as a thrombin cleavage site. In certain embodiments, the compound has a structure shown in FIG. 2 or FIG. 14. In certain embodiments, X is peptide bond or X is at least one amino acid, where A and B are each covalently bonded to X by a peptide bond. In certain embodiments, the linker is a flexible linker (e.g., (GGGGS)ₙ where n is 1, 2, or 3)), a rigid linker (e.g., PAPAP and (PT)ₙP, where n is 2, 3, 4, 5, 6, or 7), or an α-helical linker (e.g., A(EAAAK)ₙA, where n is 1, 2, 3, 4, or 5). The peptide vector may be present at the N- or C-terminal of the GDNF, BDNF, or related molecule. The invention also features a nucleic acid molecule encoding the compound, where X is a peptide bond, an amino acid, or a peptide linker. The nucleic acid may be part of a vector, and the nucleic acid may be operably linked to a promoter. The invention also features a method of making a compound by expressing a polypeptide encoded by the vector in a cell, and purifying the polypeptide. The invention also features a method of making the compound by synthesizing said compound on a solid support.

In another aspect, the invention features a method of treating (e.g., prophylactically) a subject (e.g., a human) having neurodegenerative disorder or a neuronal injury or damage by administering to the subject an effective amount of a compound of the invention. The neurodegenerative disorder may be selected from the group consisting of a polyglutamine expansion disorder, fragile X syndrome, fragile XE mental retardation, Friedreich's ataxia, myotonic dystrophy, spinocerebellar ataxia type 8, and spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis. The polyglutamine repeat disease may be Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17). The neuronal damage may be caused by an ischemic stroke, a hemorrhagic stroke, or a spinal cord injury. Other diseases that can be treated (e.g., prophylactically) using the compounds of the invention include depression and schizophrenia.

In particular embodiments of the above aspects, A is Angiopep-2 (SEQ ID NO:97), X is a peptide bond; and B is hGDNF⁷⁸⁻²¹¹, where A is joined to the N-terminal of B through X; A is Angiopep-2 (SEQ ID NO:97), X is a peptide bond, and B is hGDNF⁷⁸⁻²¹¹; where A is joined to the C-terminal of B through X; A is reversed Angiopep-2 (SEQ ID NO:117), X is a peptide bond, and B is hGDNF⁷⁸⁻²¹¹, where A is joined to the N-terminal of B through X; A is Angiopep-2 (SEQ ID NO:97), X is (GGGGS)₂, and B is hGDNF⁷⁸⁻²¹¹, where A is joined to the N-terminal of B through X; A is Angiopep-2 (SEQ ID NO:97), X is PAPAP, and B is hGDNF⁷⁸⁻²¹¹, where A is joined to the N-terminal of B through X; or A is Angiopep-2 (SEQ ID NO:97), X is A(EAAAK)₂A, and B is hGDNF⁷⁸⁻²¹¹, where A is joined to the N-terminal of B through X.

In any of the above aspects, the peptide vector may be a polypeptide substantially identical to any of the sequences set Table 1, or a fragment thereof. In certain embodiments, the peptide vector has a sequence of Angiopep-1 (SEQ ID NO:67), Angiopep-2 (SEQ ID NO:97), Angiopep-3 (SEQ ID NO:107), Angiopep-4a (SEQ ID NO:108), Angiopep-4b (SEQ ID NO:109), Angiopep-5 (SEQ ID NO:110), Angiopep-6 (SEQ ID NO:111), Angiopep-7 (SEQ ID NO:112), or reversed Angiopep-2 (SEQ ID NO:117). The peptide vector or compound of the invention may be efficiently transported into a particular cell type (e.g., any one, two, three, four, or five of liver, lung, kidney, spleen, and muscle) or may cross the mammalian BBB efficiently (e.g., Angiopep-1, -2, -3, -4a, -4b, -5, and -6). In another embodiment, the peptide vector or compound is able to enter a particular cell type (e.g., any one, two, three, four, or five of liver, lung, kidney, spleen, and muscle) but does not cross the BBB efficiently (e.g., a conjugate including Angiopep-7). The peptide vector may be of any length, for example, at least 6, 7, 8, 9, 10, 11, 12, 13, 14, 15, 16, 17, 18, 19, 20, 21, 25, 35, 50, 75, 100, 200, or 500 amino acids, or any range between these numbers. In certain embodiments, the peptide vector is 10 to 50 amino acids in length. The polypeptide may be produced by recombinant genetic technology or chemical synthesis.

TABLE 1
Exemplary Peptide Vectors

| SEQ | |
|---|---|
| ID | |
| NO: | |
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y G G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 94 | R G G R L S Y S R R F S T S T G R |
| 95 | R R L S Y S R R R F |
| 96 | R Q I K I W F Q N R R M K W K K |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |
| 117 | Y E E T K F N N R K G R S G G Y F F T |

Polypeptides Nos. 5, 67, 76, and 91, include the sequences of SEQ ID NOS: 5, 67, 76, and 91, respectively, and are amidated at the C-terminus.
Polypeptides Nos. 107, 109, and 110 include the sequences of SEQ ID NOS: 97, 109, and 110, respectively, and are acetylated at the N-terminus.

In any of the above aspects, the peptide vector may include an amino acid sequence having the formula:

X1-X2-X3-X4-X5-X6-X7-X8-X9-X10-X11-X12-X13-X14-X15-X16-X17-X18-X19

where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, at least one (e.g., 2, 3, 4, or 5) of the amino acids X1-X19 is Arg. In some embodiments, the polypeptide has one or more additional cysteine residues at the N-terminal of the polypeptide, the C-terminal of the polypeptide, or both.

In certain embodiments of any of the above aspects, the peptide vector or the GDNF, BDNF, or related molecule is modified (e.g., as described herein). The peptide or polypeptide may be amidated, acetylated, or both. Such modifications may be at the amino or carboxy terminus of the polypeptide. The peptide or polypeptide may also include peptidomimetics (e.g., those described herein) of any of the polypeptides described herein. The peptide or polypeptide may be in a multimeric form, for example, dimeric form (e.g., formed by disulfide bonding through cysteine residues).

In certain embodiments, the peptide vector or the GDNF, BDNF, or related molecule has an amino acid sequence described herein with at least one amino acid substitution (e.g., 2, 3, 4, 5, 6, 7, 8, 9, 10, 11, or 12 substitutions), insertion, or deletion or is substantially identical to an amino acid sequence described herein. The peptide or polypeptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the peptide vector may have an arginine at one, two, or three of the positions corresponding to positions 1, 10, and 15 of the amino acid sequence of any of SEQ ID NO:1, Angiopep-1, Angiopep-2, Angiopep-3, Angiopep-4a, Angiopep-4b, Angiopep-5, Angiopep-6, and Angiopep-7. In certain embodiments, the BDNF, GDNF, or related molecule may have a cysteine or lysine substitution or addition at any position (e.g., a lysine substitution at the N- or C-terminal position).

In any of the above aspects, the compound may specifically exclude a polypeptide including or consisting of any of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, the polypeptides and compounds of the invention exclude the polypeptides of SEQ ID NOs:102, 103, 104, and 105.

By “fragment” is meant a portion of a full-length amino acid or nucleic acid sequence (e.g., BDNF or GDNF). Fragments may include at least 4, 5, 6, 8, 10, 15, 20, 25, 30, 35, 40, 45, 50, 60, 70, 75, 80, 90, 100, 110, 120, 130, 140, 150, 175, 200, or 250 amino acids or nucleic acids of the full length sequence. A fragment may retain at least one of the biological activities of the full length protein.

By “substantially identical” is meant a polypeptide or nucleic acid exhibiting at least 35%, 40%, 50%, 55%, 60%, 65%, 70%, 75%, 85%, 90%, 95%, or even 99% identity to a reference amino acid or nucleic acid sequence. For polypeptides, the length of comparison sequences will generally be at least 4 (e.g., at least 5, 6, 7, 8, 9, 10, 11, 12, 13, 14, 15, 16, 17, 18, 19, 20, 25, 50, or 100) amino acids. For nucleic acids, the length of comparison sequences will generally be at least 60 nucleotides, preferably at least 90 nucleotides, and more preferably at least 120 nucleotides, or full length. It is to be understood herein that gaps may be found between the amino acids of sequences which are identical or similar to amino acids of the original polypeptide. The gaps may include no amino acids, one or more amino acids that are not identical or similar to the original polypeptide. Percent identity may be determined, for example, with n algorithm GAP, BESTFIT, or FASTA in the Wisconsin Genetics Software Package Release 7.0, using default gap weights.

By “peptide vector” is meant a compound or molecule such as a polypeptide or a peptidomimetic that can be transported into a particular cell type (e.g., liver, lungs, kidney, spleen, or muscle) or across the BBB. The vector may be attached to (covalently or not) or conjugated to an agent and thereby may be able to transport the agent into a particular cell type or across the BBB. In certain embodiments, the vector may bind to receptors present on cancer cells or brain endothelial cells and thereby be transported into the cancer cell or across the BBB by transcytosis. The vector may be a molecule for which high levels of transendothelial transport may be obtained, without affecting the cell or BBB integrity. The vector may be a polypeptide or a peptidomimetic and may be naturally occurring or produced by chemical synthesis or recombinant genetic technology.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence or severity of (e.g., preventing) a disease, disorder or condition by administering to the subject a therapeutic agent to the subject prior to the appearance of a disease symptom or symptoms.

In one example, a subject who is being treated for a particular condition is one who a medical practitioner has diagnosed as having that condition. Diagnosis may be performed by any suitable means, such as those described herein. A subject in whom the development of the condition is being treated prophylactically may or may not have received such a diagnosis. One in the art will understand that subject of the invention may have been subjected to standard tests or may have been identified, without examination, as one at high risk due to the presence of one or more risk factors.

By “subject” is meant a human or non-human animal (e.g., a mammal).

By “equivalent dosage” is meant the amount of a compound of the invention required to achieve the same molar amount of GDNF, BDNF, or related molecule in the compound of the invention, as compared to the unconjugated molecule.

By a polypeptide which is “efficiently transported across the BBB” is meant a polypeptide that is able to cross the BBB at least as efficiently as Angiopep-6 (i.e., greater than 38.5% that of Angiopep-1 (250 nM) in the in situ brain perfusion assay described in U.S. patent application Ser. No. 11/807,597, filed May 29, 2007, hereby incorporated by reference). Accordingly, a polypeptide which is “not efficiently transported across the BBB” is transported to the brain at lower levels (e.g., transported less efficiently than Angiopep-6).

By a polypeptide or compound which is “efficiently transported to a particular cell type” is meant that the polypeptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

Other features and advantages of the invention will be apparent from the following Detailed Description, the drawings, and the claims.

### BRIEF DESCRIPTION OF THE DRAWINGS

FIG. 1 shows the sequences of human GDNF and BDNF.

FIG. 2 is a schematic diagram of Angiopep2-GDNF constructs containing a His₆ tag, a thrombin cleavage site, an Angiopep-2 peptide and hGDNF⁷⁸⁻²¹¹ linked through either a peptide bond, a flexible linker, a rigid linker, or an a-helical linker.

FIG. 3 shows the sequence of the constructs described in FIG. 2.

FIGS. 4-7 show the cloning strategy for generating the GDNF constructs.

FIGS. 8-12 show the sequences of the GDNF constructs.

FIG. 13 is a schematic diagram showing the structure of Angiopep-2/GDNF bound to the GDNF family receptor α-1 (GRα-1).

FIG. 14 is a schematic diagram showing addition fusion proteins including (a) Angiopep-2 or reversed Angiopep-2 and (b) GDNF (hGDNF⁷⁸⁻²¹). Specific constructs include An2-hGDNF (N-terminal Angiopep-2 fused to hGDNF⁷⁸⁻²¹¹); hGDNF-An2 (C-terminal Angiopep-2 fused to hGDNF⁷⁸⁻²¹¹); An2NT-hGDNF (N-terminal reversed sequence Angiopep-2 fused to hGDNF⁷⁸⁻²¹¹); An2-Flex-hGDNF (N-terminal Angiopep-2 fused to hGDNF⁷⁸⁻²¹¹ through a flexible ((GGGGS)₂) linker); An2-Rig-hGDNF (N-terminal Angiopep-2 fused to hGDNF⁷⁸⁻²¹¹ through a rigid (PAPAP) linker); An2-Hel-hGDNF (N-terminal Angiopep-2 fused to hGDNF⁷⁸⁻²¹¹ through a helical (A(EAAAK)₂A) linker).

FIG. 15 is a schematic diagram showing the enzyme-linked immunosorbent assay (ELISA) used to determine whether the conjugates are capable of binding the GFRa1 receptor.

FIG. 16 is a set of graphs showing the results from the binding experiments described in FIG. 15 performed on each of the conjugates of FIG. 14.

FIG. 17 is a schematic diagram showing formation of GDNF and Angiopep-GDNF fusion protein homodimers.

FIG. 18 is a photograph of a Coomassie-stained polyacrylimide gel showing the formation of dimer in both the GDNF and the An2-GDNF polypeptides. Monomers formed when the dimers were treated with dithiothreitol (DTT).

FIG. 19 is a schematic diagram showing the GDNF signaling cascade.

FIG. 20 is a set of photographs of western blots showing that both GDNF and An2-GDNF are capable of increasing activation (phosphorylation) of components of the GDNF signaling cascade.

FIG. 21 is a graph showing results from an in situ brain perfusion assay using either GDNF or an Angiopep-2/GDNF fusion protein.

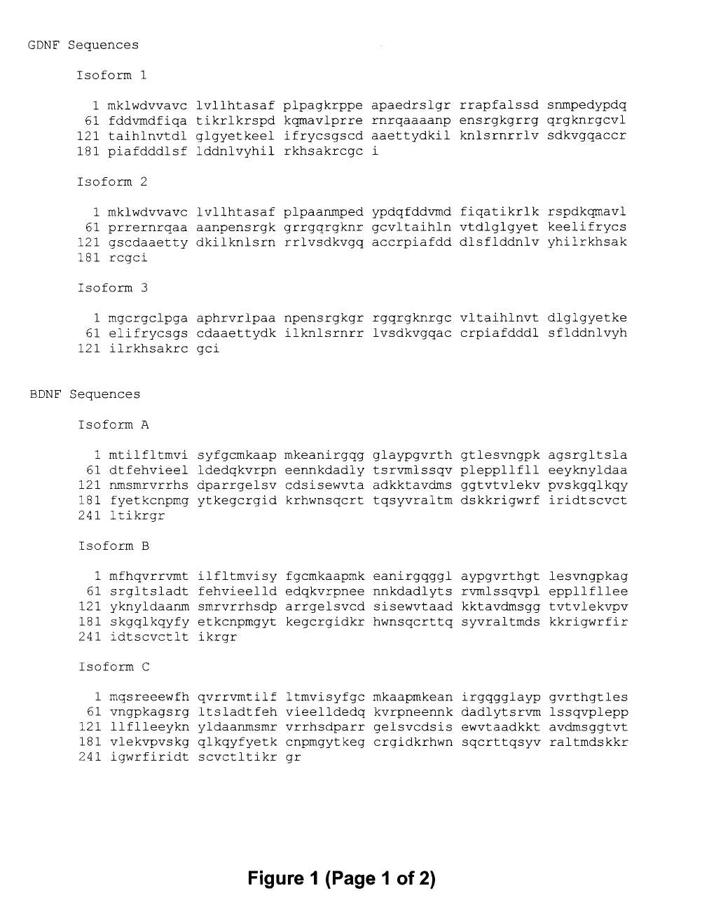

### DETAILED DESCRIPTION

We have developed compounds that include GDNF, BDNF, analogs or functional fragments thereof attached to a peptide vector capable of crossing the blood-brain barrier (BBB). These compounds can cross the BBB and thus are transported into the brain far more efficiently than GDNF, BDNF, or related molecules not attached to the peptide vector. This increased transport can result in greater efficacy, lower side effects, or a combination of the two. In cases where efficacy is increased, lower effective amounts of the compound may be administered, as compared to the GDNF, BDNF, or related molecule when not attached to the peptide vector. In other cases, where side effects are decreased, it may be possible to administer the compound at higher doses. The compounds of the invention are useful in the treatment of diseases where increased neuronal growth or a reduction of neuronal death is desired. Such diseases include neurodegenerative diseases such as Parkinson's disease (PD), amyotrophic lateral sclerosis (ALS), as well as other diseases and conditions described herein.

### GDNF and GDNF Analogs

In certain embodiments, the peptide vector is attached to GDNF, a

GDNF analog, a GDNF fragment, or a modified form thereof. In certain embodiments, the GDNF analog is a sequence substantially identical (e.g., at least 60%, 70%, 80%, 85%, 90%, 95%, 98%, 99% identical) to GDNF, a GDNF analog, or to a fragment thereof.

GDNF is secreted as a disulfide-linked homodimer, and is able to support survival of dopaminergic neurons, Purkinje cells, motoneurons, and sympathetic neurons. GDNF analogs or fragments having one or more of these activities may be used in the present invention, and activity of such analogs and fragments can be tested using any means known in the art.

Human GDNF is expressed as a 211 amino acid protein (isoform 1; SEQ ID NO:117); a 185 amino acid protein (isoform 2; SEQ ID NO:118), and a 133 amino acid protein. Mature GDNF is a 134 amino acid sequence that includes amino acids 78-211 or 118-211 of isoform 1, amino acids 92-185 of isoform 2. Isoform 3 includes a transforming growth factor like domain from amino acids 40-133.

In certain embodiments, the GDNF analog is a splice variant of GDNF. Such proteins are described in PCT Publication No. WO 2009/053536, and include the pre-(α)pro-GDNF, pre-(β)pro-GDNF, and pre-(γ)pro-GDNF splice variant, as well as the variants lacking the pre-pro region: (α)pro-GDNF, (β)pro-GDNF, and pre-(γ)pro-GDNF.

GDNF analogs are also described in U.S. Patent Application Publication No. 2009/0069230, which include a GDNF analog having the sequence:

Xaa₁-Pro-Xaa₃-Pro-Xaa₅-Xaa₆-Xaa₇-Xaa₈ (I).

where Xaa₁ is Phe, Trp, or Tyr; Xaa₃ is Leu, Ala, Ile, or Val; Xaa₅ is Ala, Leu, Ile, or Val; Xaa₆ is Gly, is any amino acid residue of the D configuration or is absent; Xaa₇ is Lys, Arg, or His or is absent; and Xaa₈ is Arg, Lys, or His or is absent. Xaa represents an amino acid, which we may also refer to as an amino acid residue. The subscripts (here, the subscripts 1-8) represent the positions of each amino acid in the peptide sequence. Thus, Xaa₁ represents the first amino acid residue in a fragment of a GDNF precursor protein.

In specific embodiments, the fragments of a GDNF precursor protein can have a sequence represented by (1) Phe-Pro-Xaa₃-Pro-Xaa₅-Xaa₆-Xaa₇-Xaa₈, (e.g., Phe-Pro-Leu-Pro-Ala-Gly-Lys-Arg); (2) Xaa₁-Pro-Leu-Pro-Xaa₅-Xaa₆-Xaa₇-Xaa₈; (3) Phe-Pro-Leu-Pro-Xaa₅-Xaa₆-Xaa₇-Xaa₈; (4) Xaa₁-Pro-Xaa₃-Pro-Ala-Xaa₆-Xaa₇-Xaa₈; (5) Phe-Pro-Xaa₃-Pro-Ala-Xaa₆-Xaa₇-Xaa₈; (6) Phe-Pro-Leu-Pro-Ala-Xaa₆-Xaa₇-Xaa₈; (7) Xaa₁-Pro-Xaa₃-Pro-Xaa₅-Gly-Xaa₇-Xaa₈; (8) Phe-Pro-Xaa₃-Pro-Xaa₅-Gly-Xaa₇-Xaa₈; (9) Phe-Pro-Leu-Pro-Xaa₅-Gly-Xaa₇-Xaa₈; (10) Phe-Pro-Leu-Pro-Ala-Gly-Xaa₇-Xaa₈; (11) Xaa₁-Pro-Xaa₃-Pro-Xaa₅-Xaa₆-Lys-Xaa₈; (12) Phe-Pro-Xaa₃-Pro-Xaa₅-Xaa₆-Lys-Xaa₈; (13) Phe-Pro-Leu-Pro-Xaa₅-Xaa₆-Lys-Xaa₈; (14) Phe-Pro-Leu-Pro-Ala-Xaa₆-Lys-Xaa₈; (15) Phe-Pro-Leu-Pro-Ala-Gly-Lys-Xaa₈; (16) Xaa₁-Pro-Xaa₃-Pro-Xaa₅-Xaa₆-Xaa₇-Arg; (17) Phe-Pro-Xaa₃-Pro-Xaa₅-Xaa₆-Xaa₇-Arg; (18) Phe-Pro-Leu-Pro-Xaa₅-Xaa₆-Xaa₇-Arg; (19) Phe-Pro-Leu-Pro-Ala-Xaa₆-Xaa₇-Arg; and (20) Phe-Pro-Leu-Pro-Ala-Gly-Xaa₇-Arg.

In another embodiment, the fragment of a GDNF precursor protein can be a fragment or portion of a GDNF precursor protein conforming to Formula I, where Xaa₁ is Phe, Xaa₃ is Leu, Xaa₅ is Ala, Xaa₆ is Gly, Xaa₇ is Lys and Xaa₈ is Arg (i.e., Phe-Pro-Leu-Pro-Ala-Gly-Lys-Arg). At least one (e.g., one, two, or three) of the amino acid residues represented by Formula I can be absent. For example, Xaa₆, Xaa₇, and/or Xaa₈ can be absent.

In another embodiment, the fragment of a GDNF precursor protein or the biologically active variants can have, or can include, a sequence of amino acid residues conforming to the amino acid sequence of Formula II:

Pro-Pro-Xaa₃-Xaa₄-Pro-Xaa₆-Xaa₇-Xaa₈-Xaa₉-Xa-a₁₀-Xaa₁₁-Xaa₁₂-Xaa₁₃-Xaa₁₄ (II)

where Xaa₃ is Glu or Asp; Xaa₄ is Ala, Gly, Ile, Leu, Met, or Val; Xaa₆ is Ala, Gly, Ile, Leu, Met, or Val; Xaa₇ is Glu or Asp; Xaa₈ is Asp or Glu; Xaa₉ is Arg, His, or Lys; Xaa₁₀ is Ser, Asn, Gln, or Thr; Xaa₁₁ is Leu, Ala, Gly, Ile, Leu, Met or Val; Xaa₁₂ is Gly, is any amino acid residue of the D-configuration, or is not present; Xaa₁₃ is Arg, His, or Lys or is not present; Xaa₁₄ is Arg, His, or Lys or is not present. An exemplary peptide conforming to Formula II can have the sequence Pro-Pro-Glu-Ala-Pro-Ala-Glu-Asp-Arg-Ser-Leu-Gly-Arg-Arg (SEQ ID NO:2).

In another embodiment, the fragments of a GDNF precursor protein or the biologically active variants can have, or can include, a sequence of amino acid residues conforming to the amino acid sequence of Formula III:

Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Xaa₁₇-Xaa₁₈-Xaa₁₉-Xaa₂₀-Xaa₂₁-Xaa₂₂ (III).

where Xaa₁ and Xaa₂ are, independently, Arg, Lys, or H is or are absent; Xaa₃ is Glu or Asp; Xaa₄ is Arg, Lys, or His; Xaa₅ is Asn, Gln, Ser, or Thr; Xaa₆ is Arg, Lys, or His; Xaa₇ is Gin, Asn, Ser, or Thr; Xaa₈, Xaa₉, Xaa₁₀, and Xaa₁₁ are, independently, Ala, Gly, Ile, Leu, Met, or Val; Xaa₁₂ is Asn, Gln, Ser, or Thr; Xaa₁₃ is Pro or Ser; Xaa₁₄ is Glu or Asp; Xaa₁₅ is Asn, Gln, Ser, or Thr; Xaa₁₆ is Ser, Asn, Gln, or Thr; Xaa₁₇ is Lys, Arg, or His; Xaa₁₈ is Gly, Ala, Ile, Leu, Met, or Val; Xaa₁₉ is Lys, Arg, or His; Xaa₂₀ is Gly, is any amino acid residue of the D-configuration, or is not present; and Xaa₂₁ and Xaa₂₂ are, independently, Arg, Lys, His, or are not present. An exemplary peptide conforming to Formula III can have the sequence Arg-Arg-Glu-Arg-Asn-Arg-Gln-Ala-Ala-Ala-Ala-Asn-Pro-Glu-Asn-Ser-Arg-Gly-Lys-Gly-Arg-Arg.

Other GDNF analogs are described in PCT Publication No. WO 2008/069876. These analogs include ERNRQAAAANPENSRGK-amide; FPLPA-amide; and PPEAPAEDRSL-amide.

Still other GDNF analogs are described in PCT Publication No. WO 2007/019860. The analogs include those having the formula:

Xₐ-(x)-X_{b}-X_{c}-X_{d}-X_{f}

wherein Xₐ is D, E, A or G, (x) is a sequence of 2-3 amino acid residues or a single amino acid residue selected from the group consisting of amino acid residues A, D, E, G, I, K, L, P, Q, S, T and V, X_{b} is amino acid residue Y or H, or a hydrophobic amino acid residue, and at least one of X_{c}, X_{d}, or X_{f} is a charged or hydrophobic amino acid residue. The analog may be 6-22 amino acids in length.

Further GDNF analogs are described in U.S. Patent Application Publication No. 2006/0258576. These analogs include FPLPA-amide, PPEAPAEDRSL-amide, LLEAPAEDHSL-amide, SPDKQMAVLP, SPDKQAAALP, SPDKQTPIFS, ERNRQAAAANPENSRGK-amide, ERNRQAAAASPENSRGK-amide, and ERNRQSAATNVENSSKK-amide.

Additional GDNF analogs can include functional fragments (e.g., any of the fragments described herein), peptides having any of the modifications described herein, or peptidomimetics thereof. Activity of such analogs and fragments can be tested using any means known in the art.

### BDNF

BNDF is glycoprotein of the nerve growth factor family of proteins. The protein is encoded as a 247 amino acid polypeptide (isoform A), a 255 amino acid polypeptide (isoform B), a 262 amino acid polypeptide (isoform C), a 276 amino acid polypeptide (isoform D), a 329 amino acid polylpeptide (isoform E). The mature 119 amino acid glycoprotein is processed from the larger precursor to yield a neutrophic factor that promotes the survival of neuronal cell populations. The mature protein includes amino acids 129-247 of the isoform A preprotein, amino acids 137-255 of the isoform B preprotein, amino acids 144-162 of isoform C preprotein, amino acids 158-276 of the isoform D preprotein, or amino acids 211 (or 212)-329 of the isoform E preprotein. BDNF acts at the TrkB receptor and at low affinity nerve growth factor receptor (LNGFR or p75). BDNF is capable of supporting neuronal survival of existing neurons and can also promote growth and differentiation of new neurons. The BDNF fragments or analogs of the invention may have any of the aforementioned activities. Activity of such analogs and fragments can be tested using any means known in the art.

BDNF analogs are described in U.S. Patent Application Publication No. 2004/0072291, which include those having a substitution of A, C, D, E, G, H, K, N P, Q R, S, or T at one more positions selected from the group consisting of 10, 16, 20, 29, 31, 36, 38, 39, 42, 44, 49, 52, 53, 54, 61, 63, 71, 76, 86, 87, 90, 92, 98, 100, 102, 103, and 105. Additional substitutions are described in Table 2 below.

TABLE 2

| Resi- | WT | |
|---|---|---|
| due | Resi- | |
| # | due | Possible substitutions |
| 9 | E | A C F G I L M P V W Y |
| 10 | L | I M F V W Y |
| 11 | S | A C F G I L M P V W Y |
| 13 | C | D E F H I K N P Q R S T V Y |
| 14 | D | A C F G I L M P V W Y |
| 15 | S | D F H I L N P Q W Y |
| 16 | I | W M Y |
| 17 | S | A C G P |
| 18 | E | T F H I P Q S |
| 19 | W | A C D E G H K N P Q R S T |
| 20 | V | W Y |
| 21 | T | D F H I L P W Y |
| 22 | A | D E H K N P Q R S T |
| 23 | A | H T |
| 24 | D | H P T |
| 28 | A | H T |
| 31 | M | W Y |
| 32 | S | A C G P |
| 34 | G | T D E H K N P Q R S |
| 35 | T | A C G P |
| 36 | V | F I L M W Y |
| 38 | V | W Y F I M |
| 39 | L | F I M V W Y |
| 41 | K | A C G H P S |
| 42 | V | I |
| 44 | V | F L M W Y |
| 45 | S | A C F P V Y |
| 46 | K | A C G P Q S T |
| 47 | G | D E H N P Q R S T |
| 48 | Q | A C G P |
| 49 | L | F I M V W Y |
| 50 | K | I P T |
| 51 | Q | A C G P |
| 52 | Y | I M V W |
| 53 | F | M W Y |
| 55 | E | A C G H N P Q S T |
| 56 | T | A C G P |
| 57 | K | A C G H P Q S T |
| 58 | C | D E G H K N P Q R S T |
| 59 | N | A C G P T |
| 60 | P | T |
| 61 | M | I V W Y |
| 87 | V | F I M W Y |
| 88 | R | A C G P |
| 89 | A | D E H K N Q R T |
| 90 | L | F I M V W Y |
| 91 | T | A C P G P |
| 92 | H | I W Y |
| 93 | D | P T |
| 94 | S | A C G P |
| 95 | K | H P |
| 96 | K | P |
| 97 | R | A C G P |
| 98 | I | H W |
| 101 | R | P T |
| 102 | F | I M V W Y |
| 103 | I | F M W Y |
| 104 | R | A C G P T |
| 105 | I | M W |
| 106 | D | A C G H I M P T |
| 107 | T | A C D E G H K N P Q S |
| 108 | S | A C D G H P |
| 109 | C | D E H K N P Q R S T |
| 110 | V | T |
| 111 | C | D E F H I K N P Q R S T V W Y |
| 112 | T | A C F G I L H P V W Y |
| 113 | L | Any amino acid |

BDNF analogs are also described in U.S. Pat. No. 6,800,607, which describes BDNF modified with 1-acyl-glycerol. These analogs include those A modified BDNF, where is the compound of the formula (1):

A(X-B)ₙ

wherein A is a residue of brain-derived neurotrophic factor, B is a residue of a 1-acyl-glycerol derivative having a hydroxyl group at the 2-position of the glycerol moiety, which is prepared by removing a hydrogen atom from the hydroxyl group, X is a chemical cross-linkage, and m is an average number of the introduction and is not less than about 0.5; (3) A modified BDNF according to the above (2), wherein X is a group of the formula (2):

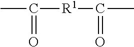

wherein R¹ is an alkylene group, or a group of the formula (3):

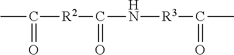

wherein R² and R³ are independently an alkylene group; (4) A modified BDNF according to the above (2), wherein the 1-acyl-glycerol derivative is 1-acyl-glycero-3-phosphoryl choline, 1-acyl-glycero-3-phosphoryl serine, or 1-acyl-grycero-3-phosphoryl ethylamine; (5) A modified BDNF according to the above (2), wherein B is a 1-acyl-glycero-3-phosphoryl choline residue of the formula (4):

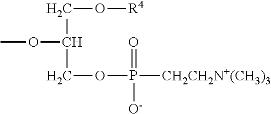

wherein R⁴ is an acyl group, a 1-acyl-glycero-3-phosphoryl serine residue of the formula (5):

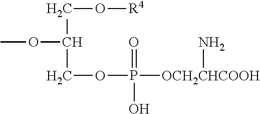

wherein R⁴ is an acyl group, or a 1-acyl-glycero-phosphoryl ethylamine residue of the formula (6):

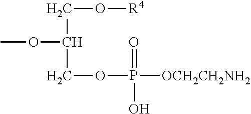

wherein R⁴ is an acyl group; (6) A modified BDNF according to the above (2) or (3), wherein B is a group of the formula (4):

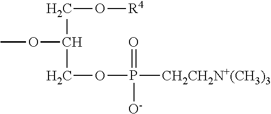

wherein R⁴ is an acyl group; (7) A modified BDNF according to any one of the above (2), (3), (4), (5) and (6), wherein the acyl group is an alkanoyl group having 8 to 30 carbon atoms; (8) A modified BDNF according to any one of the above (2), (3), (4), (5), (6) and (7), wherein the acyl group is palmitoyl group; (9) A modified BDNF according to any one of the above (2), (3), (4), (5), (6), (7) and (8), wherein m is in the range of from about 1 to about 6; (11) A modified BDNF according to the above (10), wherein R¹ is a straight chain alkylene group having 2 to 10 carbon atoms; (12) A modified BDNF according to the above (10), wherein R¹ is trimethylene.

Other BDNF analogs include those described in PCT Publication No. WO 96/15146, which described conjugates of BDNF to water soluble polymers such as polyethylene glycol. Additional BDNF analogs can include functional fragments (e.g., any of the fragments described herein), peptides having any of the modifications described herein, or peptidomimetics thereof. Activity of such analogs can be tested using any method known in the art.

### Peptide Vectors

The compounds of the invention can feature any of polypeptides described herein, for example, any of the peptides described in Table 1 (e.g., Angiopep-1 or Angiopep-2), or a fragment or analog thereof. In certain embodiments, the polypeptide may have at least 35%, 40%, 50%, 60%, 70%, 80%, 90%, 95%, 99%, or even 100% identity to a polypeptide described herein. The polypeptide may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these polypeptides (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional polypeptides may be identified by using one of the assays or methods described herein. For example, a candidate polypeptide may be produced by conventional peptide synthesis, conjugated with paclitaxel and administered to a laboratory animal. A biologically-active polypeptide conjugate may be identified, for example, based on its ability to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labeled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labelled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be as certained. Appropriate negative controls include any peptide or polypeptide known not to be efficiently transported into a particular cell type (e.g., a peptide related to Angiopep that does not cross the BBB, or any other peptide).

Additional sequences are described in U.S. Pat. No. 5,807,980 (e.g., SEQ ID NO:102 herein), U.S. Pat. No. 5,780,265 (e.g., SEQ ID NO:103), U.S. Pat. No. 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; SEQ ID NO:6; Genbank accession No. X04666). Other examples of aprotinin analogs may be found by performing a protein BLAST (Genbank: www.ncbi.nlm.nih.gov/BLAST/) using the synthetic aprotinin sequence (or portion thereof) disclosed in International Application No. PCT/CA2004/000011. Exemplary aprotinin analogs are also found under accession Nos. CAA37967 (GI:58005) and 1405218C (GI:3604747).

### Modified Polypeptides

The peptide vectors and GDNF, BDNF, or related molecule used in the invention may have a modified amino acid sequence. In certain embodiments, the modification does not destroy significantly a desired biological activity (e.g., ability to cross the BBB or neurotensin agonist activity). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified peptide or polypeptide may have or may optimize a characteristic of a polypeptide, such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy terminus of any of the polypeptides of the invention can facilitate conjugation of these polypeptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 3. If such substitutions result in a change not desired, then other type of substitutions, denominated “exemplary substitutions” in Table 3, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:

(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),

(2) neutral hydrophilic: Cysteine (Cys), Serine (Ser), Threonine (Thr)

(3) acidic/negatively charged: Aspartic acid (Asp), Glutamic acid (Glu)

(4) basic: Asparagine (Asn), Glutamine (Gin), Histidine (His), Lysine (Lys), Arginine (Arg)

(5) residues that influence chain orientation: Glycine (Gly), Proline (Pro);

(6) aromatic: Tryptophan (Trp), Tyrosine (Tyr), Phenylalanine (Phe), Histidine (His),

(7) polar: Ser, Thr, Asn, Gln

(8) basic positively charged: Arg, Lys, His, and;

(9) charged: Asp, Glu, Arg, Lys, His

Other amino acid substitutions are listed in Table 3.

TABLE 3
Amino acid substitutions

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Polypeptide Derivatives and Peptidomimetics

In addition to polypeptides consisting of naturally occurring amino acids, peptidomimetics or polypeptide analogs are also encompassed by the present invention and can form the peptide vectors or GDNF, BDNF, or related molecules used in the compounds of the invention. Polypeptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287,1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and others.

While the peptide vectors described herein may efficiently cross the BBB or target particular cell types (e.g., those described herein), their effectiveness may be reduced by the presence of proteases. Likewise, the effectiveness of the GDNF, BDNF, or related molecules used in the invention may be similarly reduced. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994). In addition to reverse-D-polypeptides, constrained polypeptides including a consensus sequence or a substantially identical consensus sequence variation may be generated by methods well known in the art (Rizo et al., Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacal. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S— alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family such as LRP or LRP2). These derivatives could act as competitive antagonists. Thus, while the present invention encompasses polypeptides or derivatives of the polypeptides described herein having an extension, desirably the extension does not destroy the cell targeting activity of the polypeptides or its derivatives.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem, Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Linkers

The GDNF, BDNF, or related molecule may be bound to the peptide vector either directly (e.g., through a covalent bond such as a peptide bond) or may be bound through a linker. Linkers include chemical linking agents (e.g., cleavable linkers) and peptides.

In some embodiments, the linker is a chemical linking agent. The GDNF, BDNF, or related molecule and peptide vector may be conjugated through sulfhydryl groups, amino groups (amines), and/or carbohydrates or any appropriate reactive group. Homobifunctional and heterobifunctional cross-linkers (conjugation agents) arc available from many commercial sources. Regions available for cross-linking may be found on the polypeptides of the present invention. The cross-linker may include a flexible arm, e.g., 2, 3, 4, 5, 6, 7, 8, 9, 10, 11, 12, 13, 14, or 15 carbon atoms. Exemplary cross-linkers include BS3 ([Bis(sulfosuccinimidyl)suberate]; BS3 is a homobifunctional N-hydroxysuccinimide ester that targets accessible primary amines), NHS/EDC (N-hydroxysuccinimide and N-ethyl-′(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups).

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible α-amine groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker includes at least one amino acid (e.g., a peptide of at least 2, 3, 4, 5, 6, 7, 10, 15, 20, 25, 40, or 50 amino acids). In certain embodiments, the linker is a single amino acid (e.g., any naturally occurring amino acid such as Cys). In other embodiments, a glycine-rich peptide such as a peptide having the sequence [Gly-Gly-Gly-Gly-Ser]ₙ where n is 1, 2, 3, 4, 5 or 6 is used, as described in U.S. Pat. No. 7,271,149. In other embodiments, a serine-rich peptide linker is used, as described in U.S. Pat. No. 5,525,491. Serine rich peptide linkers include those of the formula [X-X-X-X-Gly]_{y}, where up to two of the X are Thr, and the remaining X are Ser, and y is 1 to 5 (e.g., Ser-Ser-Ser-Ser-Gly, where y is greater than 1). In some cases, the linker is a single amino acid (e.g., any amino acid, such as Gly or Cys). Other linkers include rigid linker (e.g., PAPAP and (PT)ₙP, where n is 2, 3, 4, 5, 6, or 7) and α-helical linkers (e.g., A(EAAAK)ₙA, where n is 1, 2, 3, 4, or 5).

Examples of suitable linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. In one particular embodiment, the further linker is succinic acid which, e.g., forms an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

### Disease

Any disease or condition where enhancing neuronal survival (e.g., decreasing neuronal death rate) or increasing the rate of neuronal formation is beneficial can be treated using the compounds of the invention. Such conditions include neurodegenerative disorders, e.g., a disorder selected from the group consisting of a polyglutamine expansion disorder (e.g., Huntington's disease (HD), dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), another trinucleotide repeat expansion disorder (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer's disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis. Other conditions include injury (e.g., spinal chord injury), concussion, ischemic stroke, and hemorrhagic stroke.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that include the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject with a clinically determined predisposition or increased susceptibility to a neurological or neurodegenerative disease. Compositions of the invention can be administered to the subject (e.g., a human) in an amount sufficient to delay, reduce, or preferably prevent the onset of clinical disease. In therapeutic applications, compositions are administered to a subject (e.g., a human) already suffering from disease (e.g., a a neurological or neurodegenerative disease) in an amount sufficient to cure or at least partially arrest the symptoms of the condition and its complications. An amount adequate to accomplish this purpose is defined as a “therapeutically effective amount,” an amount of a compound sufficient to substantially improve some symptom associated with a disease or a medical condition. For example, in the treatment of a neurodegenerative disease (e.g., those described herein), an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the subject, but generally range from about 0.05 μg to about 1000 μg (e.g., 0.5-100 μg) of an equivalent amount of GDNF, BDNF or a related molecule per dose per subject. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. Because certain compounds of the invention exhibit an enhanced ability to cross the BBB, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agonist. The agents of the invention are administered to a subject (e.g. a mammal, such as a human) in an effective amount, which is an amount that produces a desirable result in a treated subject (e.g., preservation of neurons, new neuronal growth). Therapeutically effective amounts can also be determined empirically by those of skill in the art.

The subject may also receive an agent in the range of about 0.05 to 1,000 μg equivalent dose as compared to GDNF, BDNF, or the related molecule per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) μg dose per week. A subject may also receive an agent of the composition in the range of 0.1 to 3,000 μg per dose once every two or three weeks.

Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

### EXAMPLE 1

### Angiopep-2/GDNF Constructs

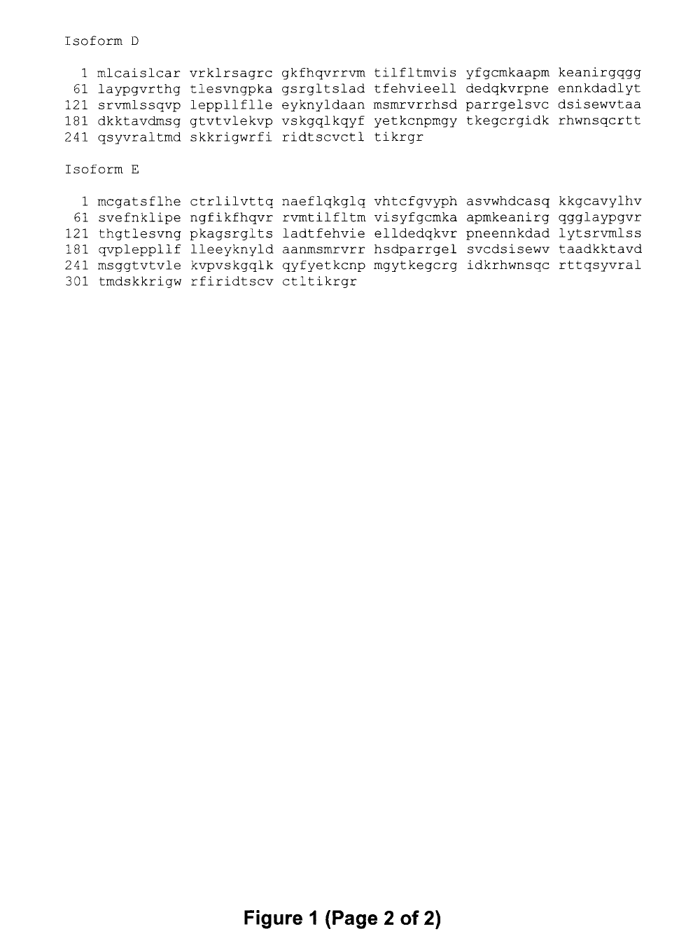

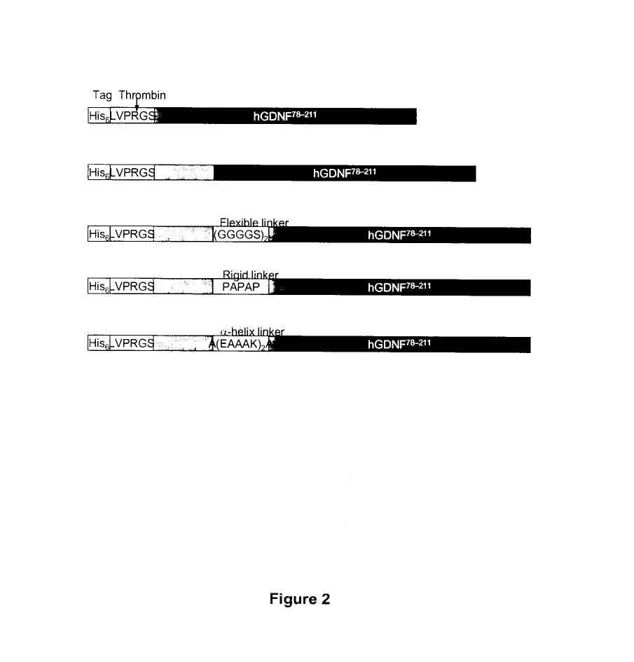

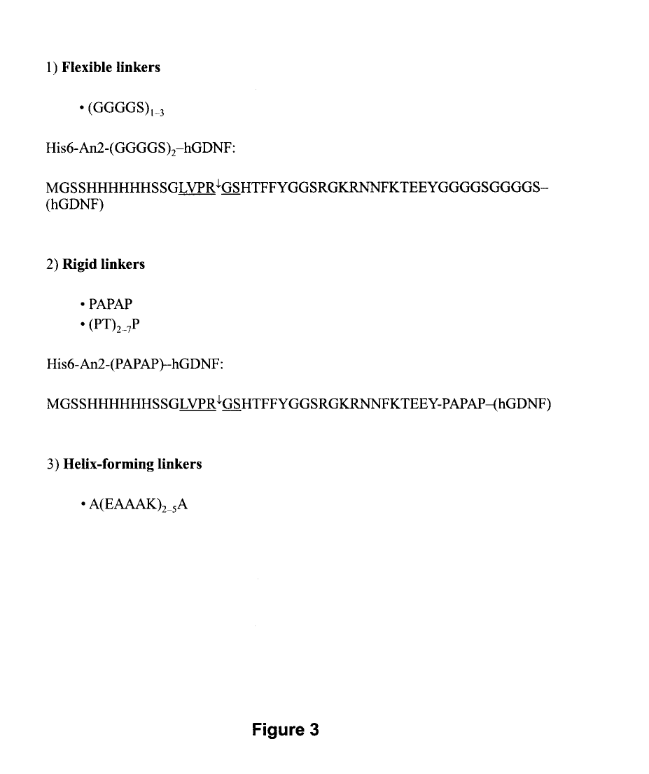

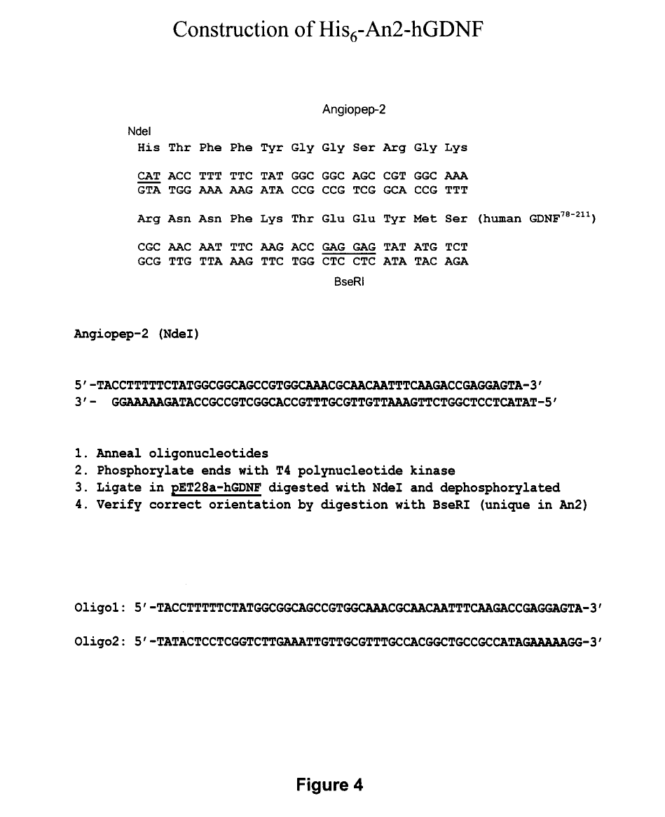

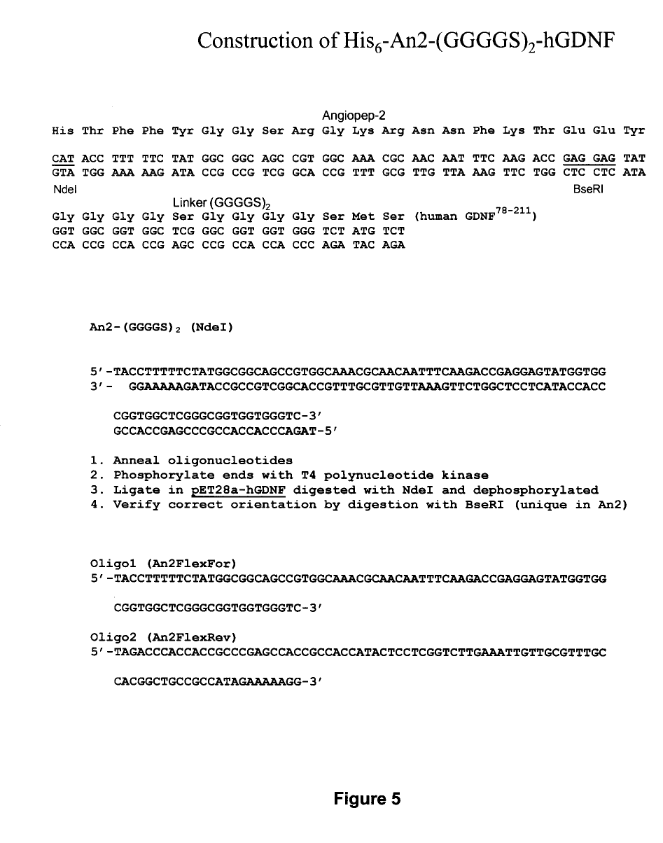

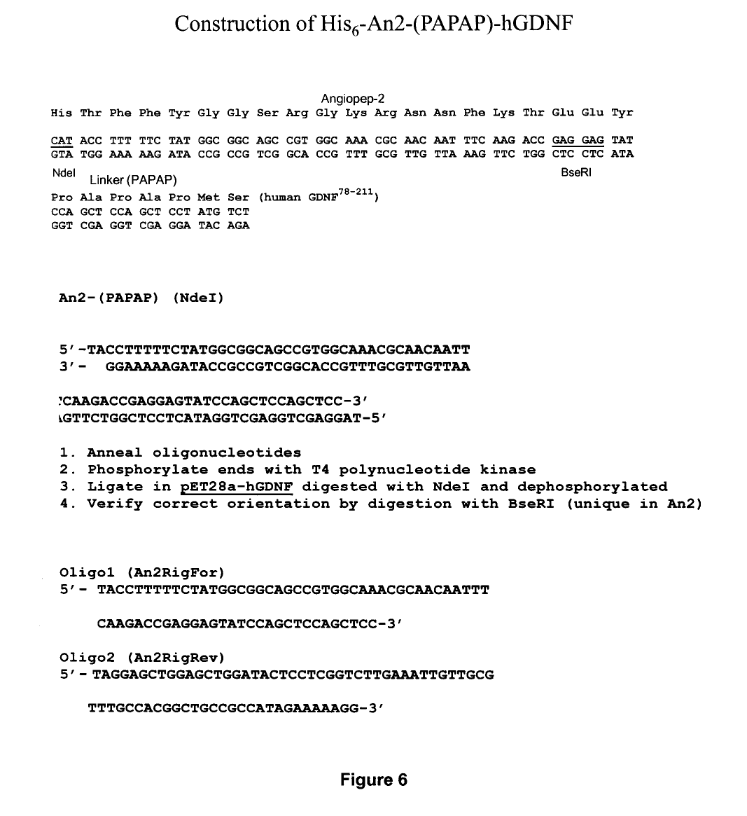

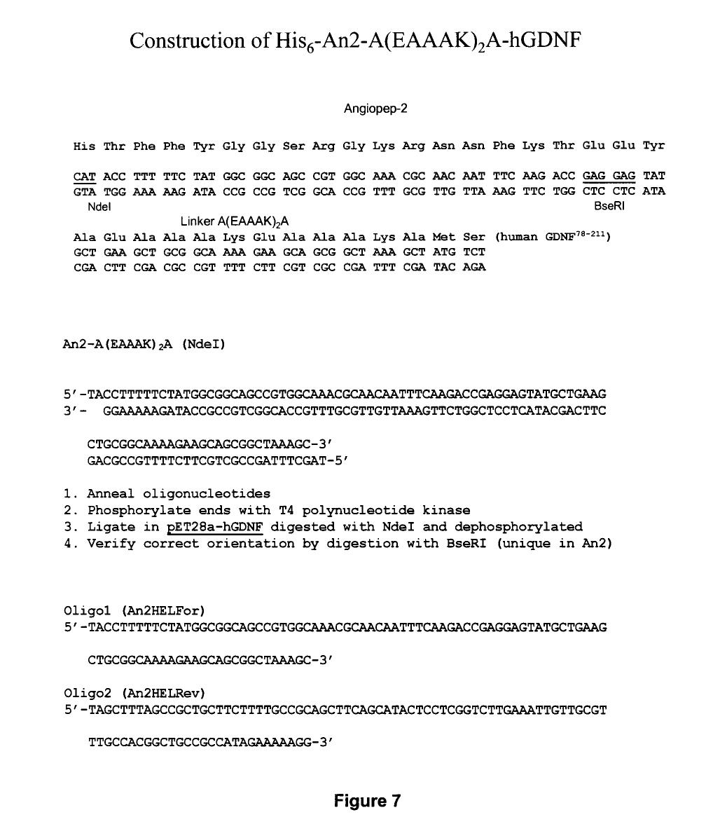

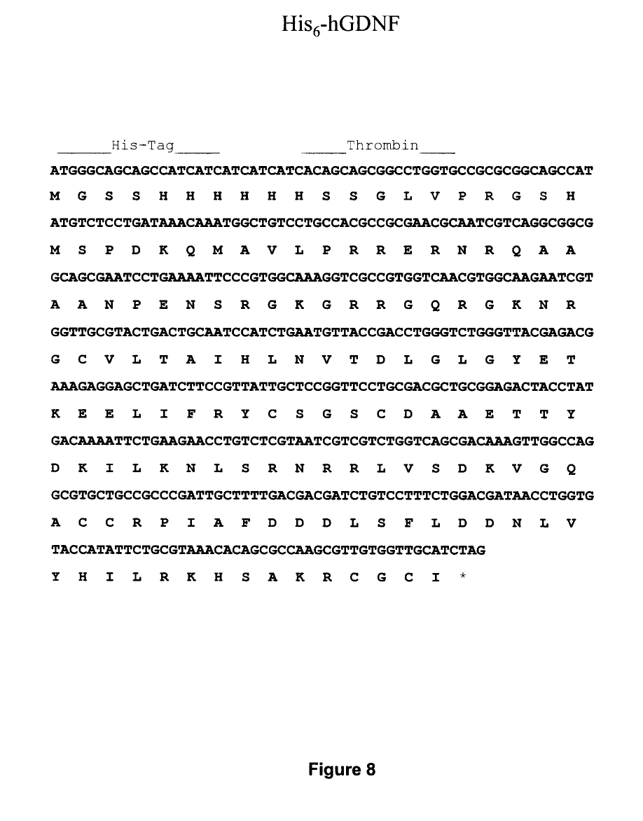

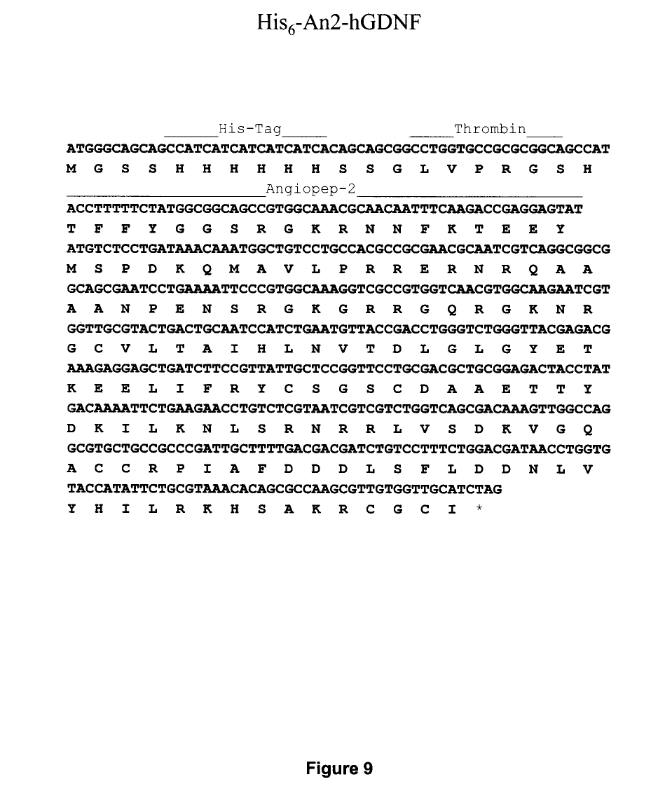

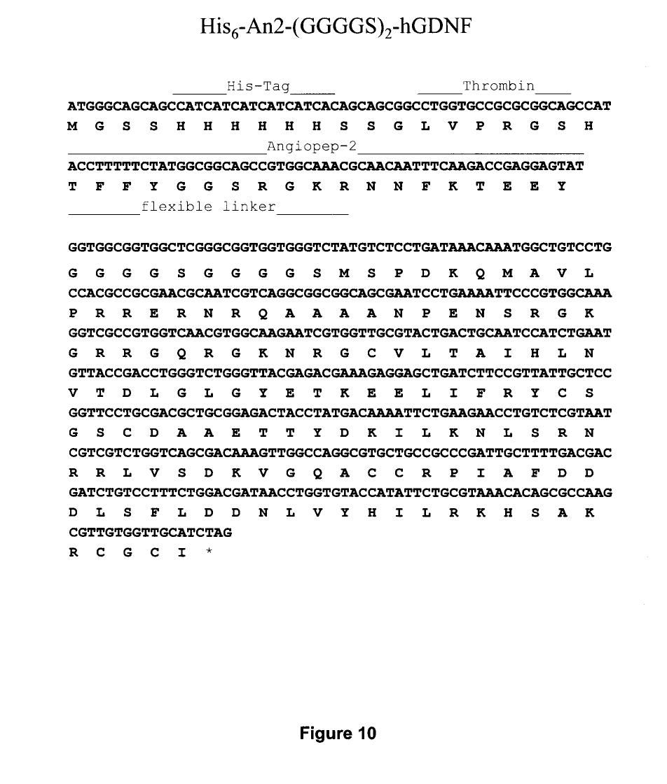

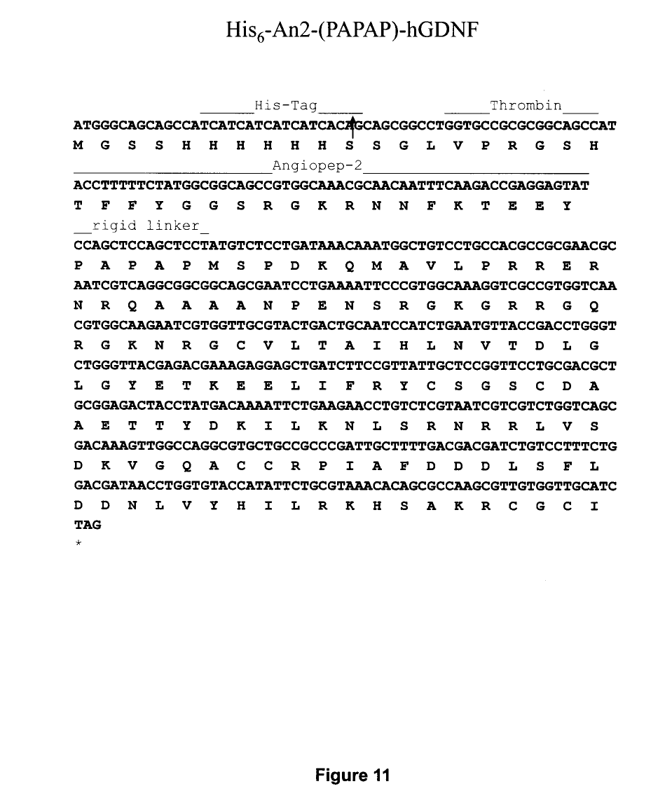

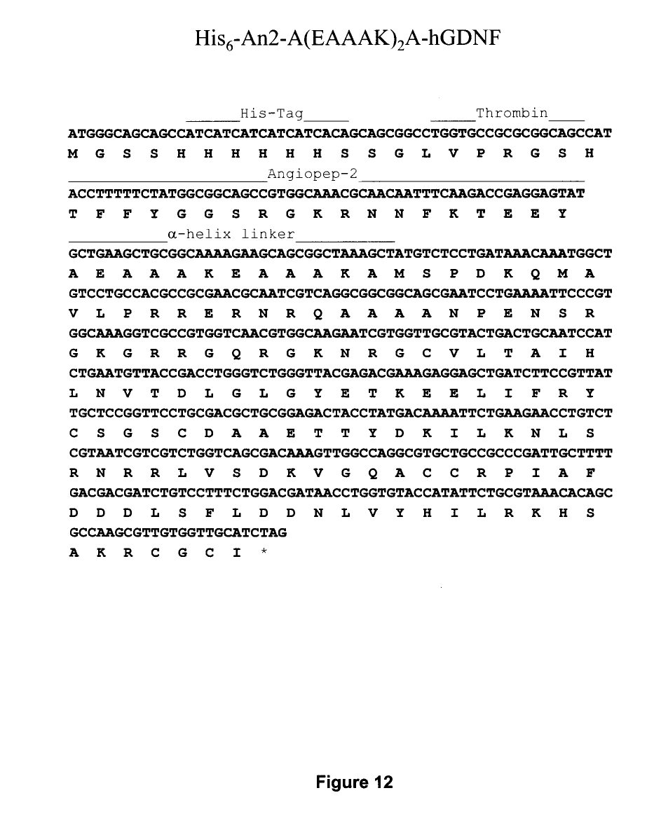

Constructs including the Angiopep-2 and hGDNF sequences (hGDNF⁷⁸⁻²¹¹) are generated. These constructs include an N-terminal (His)₆ tag, a thrombin cleavage site, the Angiopep-2 sequence, and the GDNF sequence. A control peptide, lacking the Angiopep-2 sequence is also generated (FIG. 2). The amino acid sequences of the N-terminal portion of these sequences are shown in FIG. 3. The strategy for cloning these constructs is described in FIGS. 4-7. A similar strategy can be employed to generated BDNF constructs. Sequences of the constructs are shown in FIGS. 8-12. An image showing an Angiopep-2-GDNF compound bound to the GFRα1 is shown in FIG. 13.

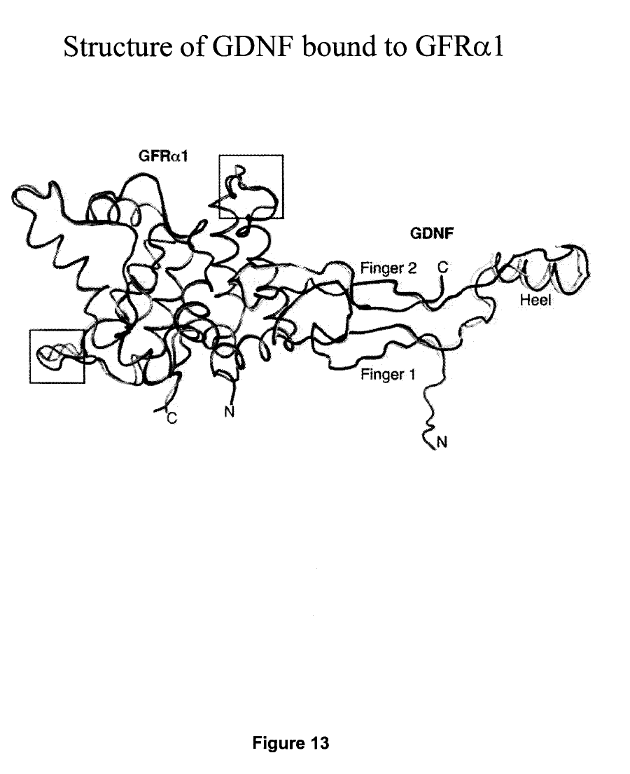

Additional GDNF constructs were generated, as shown in FIG. 14. These include an hGDNF⁷⁸⁻²¹¹ with Angiopep-2 attached at its N-terminus (An2-hGDNF); hGDNF⁷⁸⁻²¹¹ with Angiopep-2 attached at its C-terminus (hGDNF-An2); hGDNF⁷⁸⁻²¹¹ with reversed Angiopep-2 attached at its N-terminus (An2NT-hGDNF); hGDNF⁷⁸⁻²¹¹ with Angiopep-2 attached at its N-terminus through a flexible ((GGGGS)₂) linker (An2-Flex-hGDNF); hGDNF⁷⁸⁻²¹¹ with Angiopep-2 attached at its N-terminus through a rigid (PAPAP) linker (An2-Rig-hGDNF); and hGDNF⁷⁸⁻²¹¹ with Angiopep-2 attached at its N-terminus through a helical (A(EAAAK)₂A) linker (An2-Hel-hGDNF).

### EXAMPLE 2

### Receptor Binding of GDNF Conjugates

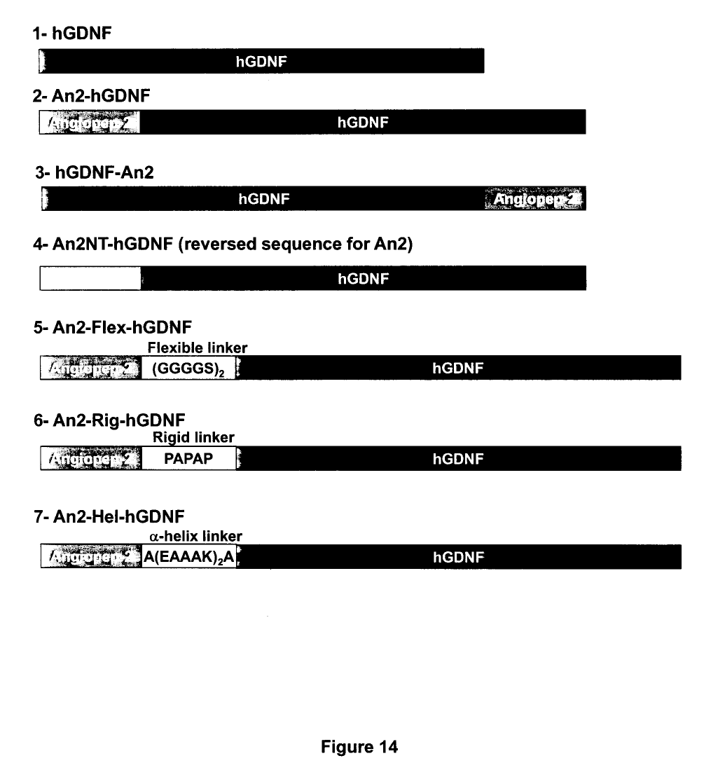

To measure receptor binding of GDNF conjugates, a double sandwich Elisa was used. Briefly, mouse anti-human IgG antibodies were bound to a plate (FIG. 15). A GFRα1 receptor/IgG Fc fusion was added, which bound to the antibodies. To measure ligand binding, GDNF, a GDNF conjugate, or Angiopep-2 were each added to the plates. The plates were then treated sequentially with a goat anti-GDNF antibody and an alkaline phosphatase-conjugated rabbit anti-goat IgG antibody. The samples were then treated with p-nitrophenyl phosphate (p-NPP), an alkaline phosphatase (AP) substrate that changes from colorless to yellow upon AP treatment. Binding of the proteins was measured on the basis of this color change.

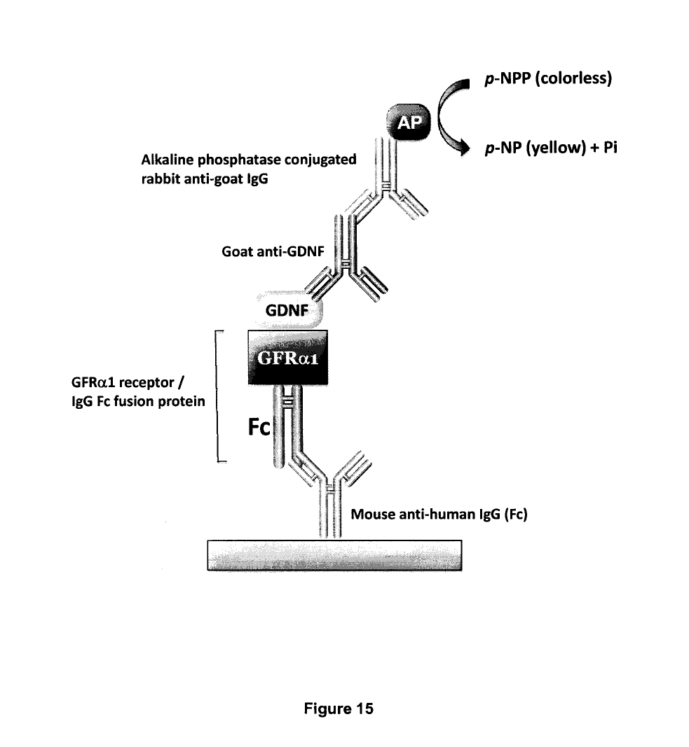

In this assay, all of the fusion proteins tested were capable of binding the GDNF receptor at levels similar to that of the GDNF by itself (FIG. 16). Angiopep-2, a negative control, was not observed to bind the receptor. Binding constants and for each for each protein were calculated, as shown in the table below. As can be seen, all of the fusion proteins were able to bind the GDNF receptor effectively.

| | Kd | Bmax |
|---|---|---|
| | (nM) | (A₄₀₅/min) × 10⁻³ |
| GDNF | 0.24 | 155 |
| An2-GDNF | 0.68 | 146 |
| GDNF-An2 | 0.53 | 155 |
| An2NT-GDNF | 0.31 | 161 |
| An2-Flex-GDNF | 0.16 | 161 |
| An2-Rig-GDNF | 0.82 | 202 |
| An2-Hel-GDNF | 0.27 | 142 |
| An2 | ND | ND |

### EXAMPLE 3

### Formation of Homodimers

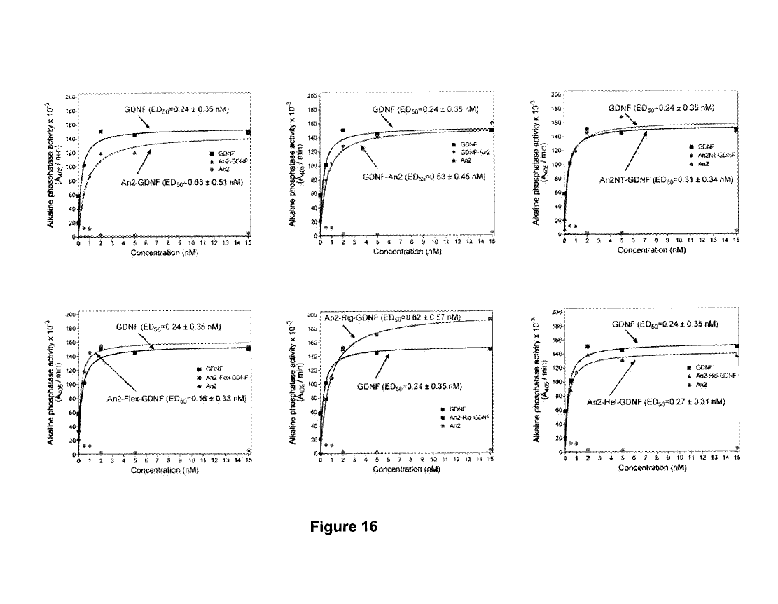

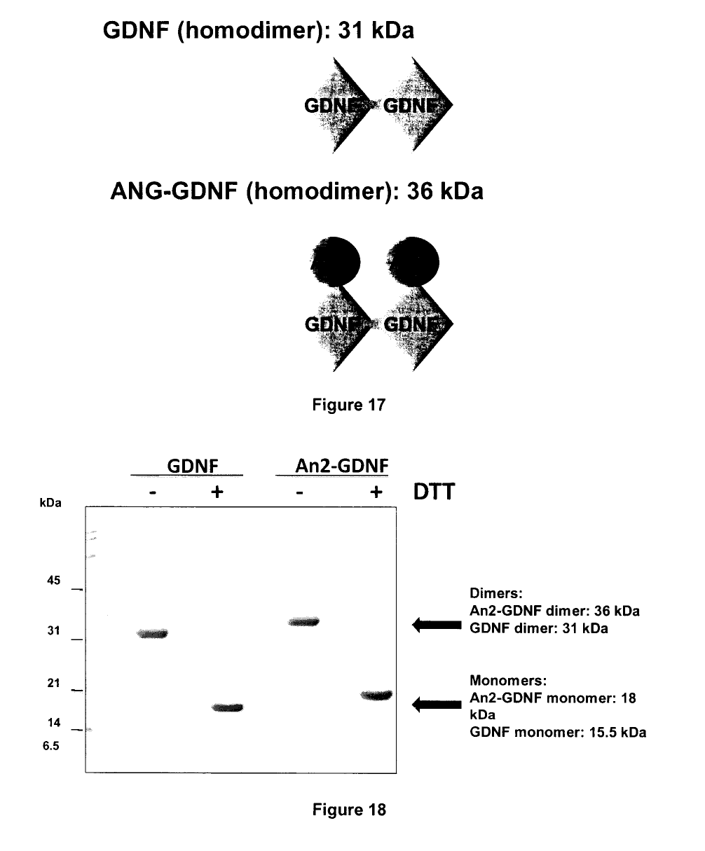

As described above, GDNF is known to form homodimers through disulfide bonds (FIG. 17). Formation of such homodimers was also observed with the An2-GDNF protein (FIG. 18). By treatment with a reducing agent such as dithiolthreitol (DTT), these disulfide bonds could be reduced.

### EXAMPLE 4

### Activation of the GDNF Signaling Cascade

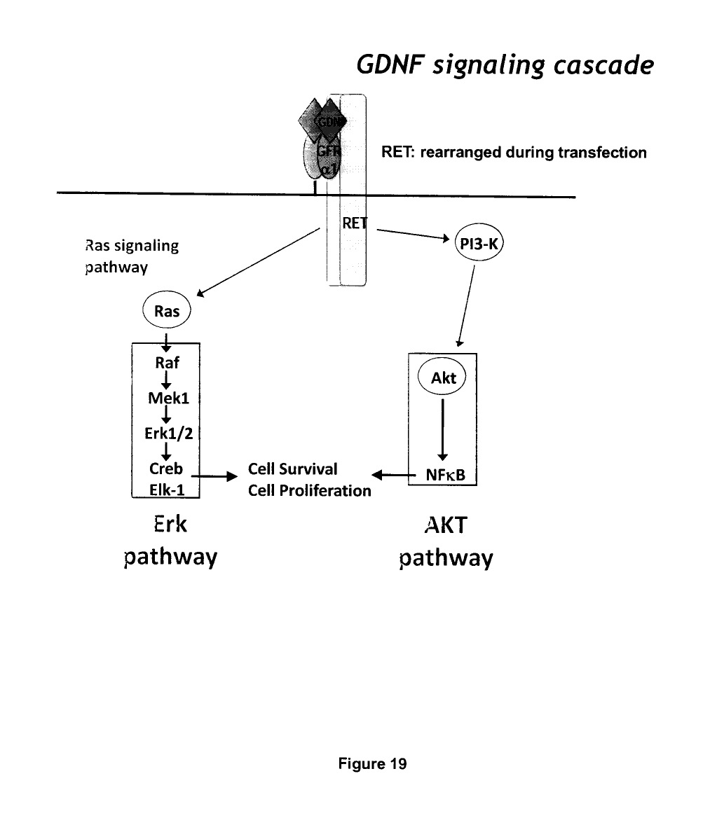

As explained above, GDNF binds to the GFRα1 receptor. The ligand-receptor complex then binds to the tyrosine kinase receptor RET. This receptor can then activate two pathways, an Akt pathway through phosphatidylinositol 3-kinase (PI3K) and the Erk pathway through Ras. Activation of each of these pathways results in increased cell survival and proliferation (FIG. 19).

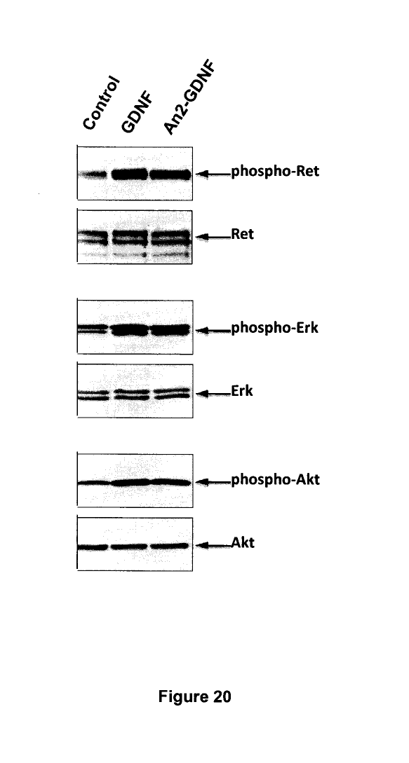

To test whether the fusion proteins were capable of activating these pathways, cells were treated for ten minutes with GDNF, An2-GDNF, or were untreated. From these experiments, increased in phosphorylated RET, phosphorylated Erk, and phosphorylated Akt were observed (FIG. 20) using both GDNF and An2-GDNF. These results indicate that An2-GDNF, like GDNF, was capable of activating the GDNF pathways.

### EXAMPLE 5

### In Situ Brain Perfusion

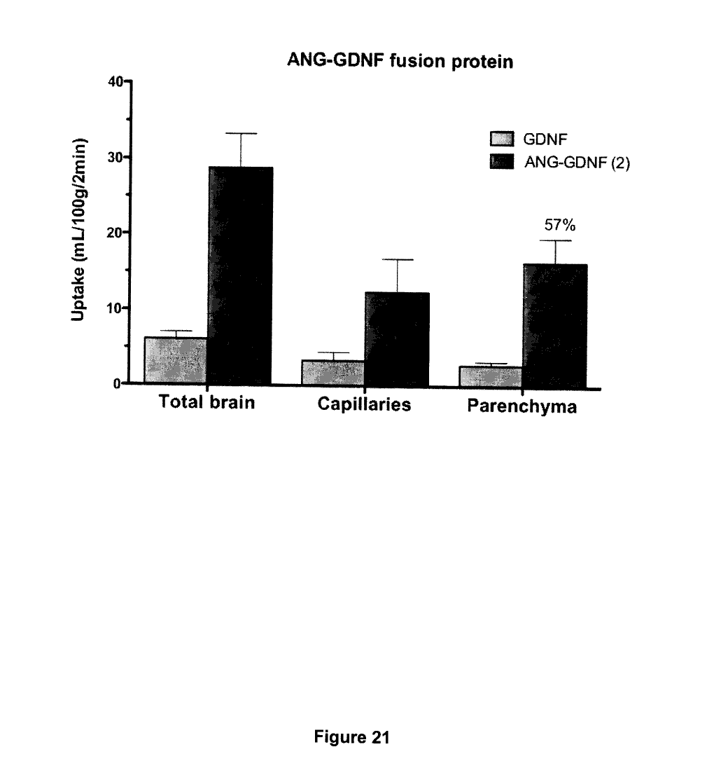

To determine whether the GDNF fusion proteins were able to cross the blood-brain barrier, an in situ perfusion assay was performed. Such assays are described, for example, in PCT Publication WO 2006/086870. From these results the Angiopep-2-GDNF conjugate was observed to cross the BBB far more effectively than unconjugated GDNF (FIG. 21).

### Other Embodiments

All patents, patent applications including U.S. Provisional Application No. 61/186,246, filed Jun. 11, 2009, and publications mentioned in this specification are herein incorporated by reference to the same extent as if each independent patent, patent application, or publication was specifically and individually indicated to be incorporated by reference.

1. A compound comprising the formula:
A-X-B
wherein A is peptide vector; B is a polypeptide substantially identical to:
(i) GDNF, a fragment thereof having at least one GDNF activity, or a GDNF analog; or
(ii) BDNF, a fragment thereof having at least one BDNF activity, or a BDNF analog; and
X is a linker joining A to B.

2. The compound of claim 1, wherein said compound is capable of crossing the blood-brain barrier.

3. The compound of claim 1, wherein said B comprises a mature form of GDNF or BDNF.

4. The compound of claim 1, wherein A comprises an amino acid sequence at least 70% identical to a sequence selected from the group consisting of Angiopep-2 (SEQ ID NO:97), reversed Angiopep-2 (SEQ ID NO:117), Angiopep-1 (SEQ ID NO:67), cys-Angiopep-2 (SEQ ID NO:113), and Angiopep-2-cys (SEQ ID NO:114).

5. The compound of claim 4, wherein said sequence identity is at least 90%.

6. The compound of claim 5, wherein A comprises or consists of an amino acid sequence selected from the group consisting of Angiopep-2 (SEQ ID NO:97), reversed Angiopep-2 (SEQ ID NO:117), Angiopep-1 (SEQ ID NO:67), cys-Angiopep-2 (SEQ ID NO:113), and Angiopep-2-cys (SEQ ID NO:114).

7-8. (canceled)

9. The compound of claim 1, wherein X is a peptide bond or is at least one amino acid; and A and B are each covalently bonded to X by a peptide bond.

10. The compound of claim 9, wherein X is selected from the group consisting of (GGGGS)ₙ, where n is 1, 2, or 3; PAPAP; (PT)ₚP, where p is 2, 3, 4, 5, 6, or 7; and A(EAAAK)_{q}A, where q is 1, 2, 3, 4, or 5.

11. The compound of claim 1, wherein A is Angiopep-2 (SEQ ID NO:97); X is a peptide bond; and B is hGDNF⁷⁸⁻²¹¹; wherein A is joined to the N-terminal of B through X.

12. The compound of claim 1, wherein A is Angiopep-2 (SEQ ID NO:97); X is a peptide bond; and B is hGDNF⁷⁸⁻²¹¹; wherein A is joined to the C-terminal of B through X.

13. The compound of claim 1, wherein A is reversed Angiopep-2 (SEQ ID NO:117); X is a peptide bond; and B is hGDNF⁷⁸⁻²¹¹; wherein A is joined to the N-terminal of B through X.

14. The compound of claim 1, wherein A is Angiopep-2 (SEQ ID NO:97); X is (GGGGS)₂; and B is hGDNF⁷⁸⁻²¹¹; wherein A is joined to the N-terminal of B through X.

15. The compound of claim 1, wherein A is Angiopep-2 (SEQ ID NO:97); X is PAPAP; and B is hGDNF⁷⁸⁻²¹¹; wherein A is joined to the N-terminal of B through X.

16. The compound of claim 1, wherein A is Angiopep-2 (SEQ ID NO:97); X is A(EAAAK)₂A; and B is hGDNF⁷⁸⁻²¹¹; wherein A is joined to the N-terminal of B through X.

17. A nucleic acid molecule encoding the compound of claim 9.

18. A vector comprising the nucleic acid molecule of claim 17, wherein said nucleic acid is operably linked to a promoter.

19. A method of making a compound, said method comprising expressing a polypeptide encoded by the vector of claim 18 in a cell, and purifying said polypeptide.

20. A method of making a compound of claim 9, said method comprising synthesizing said compound on a solid support.

21. A method of treating a subject having a neurodegenerative disorder, said method comprising administering to said subject an effective amount of a compound of claim 1.

22. The method of claim 21, wherein said neurodegenerative disorder is selected from the group consisting of a polyglutamine expansion disorder, fragile X syndrome, fragile XE mental retardation, Friedreich's ataxia, myotonic dystrophy, spinocerebellar ataxia type 8, and spinocerebellar ataxia type 12, Alexander disease, Alper's disease, Alzheimer's disease, amyotrophic lateral sclerosis (ALS), ataxia telangiectasia, Batten disease (Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

23. The method of claim 22, wherein said polyglutamine repeat disease is Huntington's disease (HD), dentatorubropallidoluysian atrophy, Kennedy's disease (also referred to as spinobulbar muscular atrophy), or a spinocerebellar ataxia selected from the group consisting of type 1, type 2, type 3 (Machado-Joseph disease), type 6, type 7, and type 17).

24. The method of claim 21, wherein said subject is a human.

25. A method of treating a subject having a neuronal damage, depression, or schizophrenia, said method comprising administering to said subject an effective amount of a compound of claim 1.

26. The method of claim 25, wherein said neuronal damage is caused by an ischemic stroke, a hemorrhagic stroke, or a spinal cord injury.

27. The method of claim 25, wherein said subject is a human.

28-31. (canceled)
